(19) United States
(12) Patent Application Publication
(10) Pub. No.: US 2014/0287025 A1
(43) Pub. Date: Sep. 25, 2014

(54) Sustained Release Composition For Local Injection to Treat Eye Diseases

(71) Applicants: Taiwan Liposome Company, Taipei (TW); TLC Biopharmaceuticals, Inc., South San Francisco, CA (US)

(72) Inventors: Jun-Jen LIU, Taipei (TW); Yun-Long Tseng, Taipei (TW); Sheue-Fang SHIH, Taipei (TW); Luke S.S. GUO, South San Francisco, CA (US); Keelung HONG, South San Francisco, CA (US)

(73) Assignees: Taiwan Liposome Company, Taipei (TW); TLC Biopharmaceuticals, Inc., South San Francisco, CA (US)

(21) Appl. No.: 14/248,633

(22) Filed: Apr. 9, 2014

Related U.S. Application Data
(63) Continuation of application No. 11/985,630, filed on Nov. 16, 2007.
Application No. 11/985,630 is a continuation of application No. 14/200,439, filed on Mar. 7, 2014.
(60) Provisional application No. 60/866,121, filed on Nov. 16, 2006.

(51) Int. Cl. A61K 9/00; A61K 47/48

(52) U.S. Cl. CPC A61K 9/0048; A61K 47/48215
USPC 424/450; 424/178.1

(57) ABSTRACT

The invention provides a sustained release composition for intravitreal injection to the eye of a subject in need thereof. The sustained release composition contains an effective amount of a therapeutic agent in association with a nanosphere. The nanosphere contains a particle that comprises a particle-forming component capable of forming a vesicle, and an agent-carrying component capable of forming a complex with the therapeutic agent via electrostatic charge-charge interaction or hydrophobic-hydrophobic interaction. The particle-forming component has at least one head group moiety selected from a hydrophobic head group moiety, a polar head group moiety and a combination thereof. The agent-carrying component is a chemical entity that contains one or more negatively or positively charged groups.

### CROSS REFERENCE TO RELATED APPLICATIONS

This application is a continuation application of U.S. application Ser. No. 11/985,630, filed Nov. 16, 2007, which claims the benefit of U.S. Provisional Application No. 60/866,121, filed Nov. 16, 2006. This application is also a continuation application of U.S. application Ser. No. 14/200,439, filed Mar. 7, 2014. The disclosures of all of the above-identified applications are incorporated by reference herein in their entirety.

### BACKGROUND OF THE INVENTION

The present invention is generally related to drug delivery, and more particularly related to a sustained release composition for local injection to treat eye diseases.

Eye diseases, such as age-related macular degeneration (AMD) and diabetic retinopathy (DR) that occur at the back of eyes are the leading causes of blindness in the elderly and many productive individuals in the developed country (Aiello, L. M. (2003) Am. J. Ophthalmol. 136, 122-135; Klein, R. et al. (1992) Ophthalmology 99, 933-943). For an effective therapy, it is essential that a therapeutic concentration of the pharmacological agent is present at the disease site for an extended time.

Eye is an enclosed organ of the body. The blood circulation through the eye is slower than the rest of the body. Delivery of an effective dose of the drug to the eye, particularly the rear end of the eye, such as retinal or choroidal tissues, remains a difficult task. Current methods for ocular drug delivery include topical administration (eye drops), systemic administration (oral or intravenous), subconjunctival injection, periocular injection, intravitreal injection, and surgical implant. Intravitreal injection has been proposed as an efficient way to deliver therapeutic agents to the posterior portion of the eye, in close proximity to the retina. Since the dosage of the therapeutic agents delivered to the eye may decline over time, patients usually have to receive frequent intravitreal injections in order to ensure sufficient amount for the eye treatment. A high frequency of the intravitreal injection, an invasive procedure, often intimidates or discourages the patients to approach for the treatment. Therefore, it is desirable to develop a system for releasing a therapeutic agent in a sustained period to the eyes.

### BRIEF SUMMARY OF THE INVENTION

One aspect of the present invention relates to a sustained release composition for intravitreal injection to the eye of a subject in need thereof. The sustained release composition comprises an effective amount of a therapeutic agent in association with a nanosphere. The nanosphere comprises a particle-forming component that is able to form a vesicle, and an agent-carrying component that is able to form a complex with the therapeutic agent via electrostatic charge-charge interaction or hydrophobic-hydrophobic interaction. The particle-forming component has hydrophobic and polar head group moieties alone or in combination. The agent-carrying component is a chemical entity that contains one or more negatively or positively charged groups. After intravitreal injection of a sustained release composition according to an embodiment of the present invention at a disease site, a therapeutic agent can be released from the sustained release composition at the disease site for more than 7 days, and more preferably 28 days.

Another aspect of the present invention relates to a method for providing a sustained supply of a therapeutic agent in the eye of a subject in need thereof. The method comprises:
providing a sustained release composition according to an embodiment of the invention; and
intravitreally injecting the sustained release composition to a site of the eye in the subject.

Additional aspects and advantages of the present invention will be set forth in part in the description which follows, and in part will be apparent from the description, or may be learned by practice of the invention. The aspects and advantages of the invention will be realized and attained by means of the elements and combinations as described.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments, which are presently preferred. It should be understood, however, that the invention is not limited to the precise arrangements and instrumentalities shown.

In the drawings:

FIG. 1 shows single-photon emission computed tomography (SPECT) images of rats after intravitreal injection of radio-labeled (A) fragmented antibody (Fab), (B) whole antibody (Ab), and (C) Fab-nanosphere according to one preferred embodiment of the invention; and

FIG. 2 illustrates the retention percentage-time profiles of Fab and Fab-nanosphere in vitreous humor post 75 μg/eye ITV administration.

### DETAILED DESCRIPTION OF THE INVENTION

Unless defined otherwise, all technical and scientific terms used herein have the same meaning as commonly understood by one of ordinary skill in the art to which the invention pertains. Although any methods and materials similar or equivalent to those described herein can be used in the practice of testing of the present invention, the preferred materials and methods are described herein.

As used herein, the article “a” or “an” means one or more than one (that is, at least one) of the grammatical object of the article, unless otherwise made clear in the specific use of the article in only a singular sense.

The present invention relates to a sustained release composition for intravitreal injection to the eye of a subject in need thereof. The sustained release composition comprises an effective amount of a therapeutic agent in association with a nanosphere. The nanosphere comprises a particle-forming component that is able to form a vesicle, and an agent-carrying component that is able to form a complex with the therapeutic agent via electrostatic charge-charge interaction or hydrophobic-hydrophobic interaction; wherein the particle-forming component has hydrophobic and polar head group moieties alone or in combination; and the agent-carrying component is a chemical entity that contains one or more negatively or positively charged groups.

According to an embodiment of the invention, the local intravitreal injection of a sustained release composition according to an embodiment of the present invention to a disease site of the eye results in accumulation of a therapeutic agent at the disease site for more than 7 days after the administration. In another embodiment of the invention, the therapeutic agent is accumulated at the disease site for more than 28 days after the administration.

The term “nanosphere” as used herein refers to a particle comprising a particle-forming component that is able to form a vesicle, and an agent-carrying component that is able to form a complex with the therapeutic agent via electrostatic charge-charge interaction or hydrophobic-hydrophobic interaction The nanosphere or particle has a mean size of the diameter of about 30 to 500 nm.

### Particle-Forming Component

The term “particle-forming component” as used herein refers to a component that is able to form able to form a vesicle, which can have a hydrophobic head group moiety, a polar head group moiety, alone or in combination, such as an amphipathic lipid or a hydrophilic polymer. The amphipathic lipid may be a phospholipid, selected from the group consisting of egg phosphatidyl choline (EPC), hydrogenated egg phosphatidyl choline (HEPC), soy phosphatidyl choline (SPC), hydrogenated soy phosphatidyl choline (HSPC), dipalmitoyl phosphatidyl choline (DPPC) and distearyloyl phosphatidyl choline (DSPC), diarachidoyl phosphatidyl choline, dimyristoyl phosphatidyl ethanolamine (DMPE), dipalmitoyl phosphatidyl ethanolamine (DPPE), distearoyl phosphatidyl ethanolamine (DSPE), diarachidoyl phosphatidyl ethanolamine (DAPE), and dipalmitoyl phosphatidyl glycerol (DPPG). In one example of the invention, the phospholipid is distearyloyl phosphatidyl choline (DSPC). In another example of the invention, the phospholipid is cholesterol.

The particle-forming component may be a hydrophilic polymer having a long chain highly hydrated flexible neutral polymers attached to lipid molecules. Examples of the hydrophilic polymer include, but are not limited to, polyethylene glycol (PEG), polyethylene glycol derivatized with Tween®, polyethylene glycol derivatized with distearoylphosphatidylethanolamine (PEG-DSPE), ganglioside GM₁, and synthetic polymers. In accordance with one embodiment of the invention, the hydrophilic polymer is PEG having a molecular weight of about 500 to 5000 daltons. In one preferred embodiment, the particle-forming component may be PEG having a molecular weight of approximately 2000, such as 1,2-Distearoyl-sn-Glycero-3-Phosphoethanolamine-N-[Maleimide(Polyethylene Glycol)2000].

### Agent-Carrying Component

The term “agent-carrying component” used herein refers to a chemical entity that contains one or more negatively or positively charged groups having hydrophobic and polar head group moieties alone or in combination. The agent-carrying component can be any suitable chemical entity that contains one or more negatively or positively charged groups. The chemical entity may be charged by deprotonation to a negative charged agent-carrying component or by protonation to a positive charged agent-carrying component. The agent-carrying component is able to form a complex with the therapeutic agent via electrostatic charge-charge interaction or hydrophobic-hydrophobic interaction.

The negatively charged agent-carrying component may be a divalent anion, a trivalent anion, a polyvalent anion, a polymeric polyvalent anion, a polyanionized polyol, or a polyanionized sugar. Examples of the divalent and trivalent anions include, but are not limited to, sulfate, phosphate, pyrophophosphate, tartrate, succinate, maleate, borate, and citrate. The polyanionic polymer has an organic or inorganic backbone, and a plurality of anionic functional groups. Examples of the polyanionic polymers include but are not limited to polyphosphate, polyvinylsulfate, polyvinylsulfonate, polycarbonate, acidic polyaminoacids and polynucleotides.

The positively charged agent-carrying component, described in the present invention, can be any organic polycationics such as polyamines, polyammonium molecules, and basic polyamino acids. A preferred polyamine includes spermidine and spermine. Small polycationic molecules are known to condense nucleic acids via electrostatic charge-charge interactions (Plum, G. E. et al. (1990) Biopolymers 30, 631-643). The positively charged agent-carrying component can also be amphiphilic cationic lipids that carry a net positive charge at physiological pH. Such lipids include, but are not limited to, dioleoyldimethylammonium chloride (DODAC), N-[2,3-(dioleyloxy)propyl]-N,N,N-trimethylammonium chloride (DOTMA), dimethyldioctadecylammonium bromide (DDAB), 1,2-dioleoyl-3-trimethylammonium-propane (DOTAP), 3β-[N-(N′,N′-dimethylaminoethane)-carbamoyl]-cholesterol hydrochloride (DC-Chol) and 1,2-dimyristyloxypropyl-3-dimethyl-hydroxy ethyl ammonium bromide (DMRIE). The amphiphilic cationic lipids may participate or assist the particle-forming component to form a surrounding lipid barrier of the particle.

In addition, the agent-carrying component can be a chelating agent that forms chelating complex with a divalent or trivalent cation, including a transition metal, such as nickel, indium, iron, cobalt, calcium, magnesium ions. Examples of the chelating agents include, but are not limited to, thylenediaminetetraacetic acid (EDTA), diethylenetriaminepentaacetic acid (DTPA), nitroltriacetic acid (NTA), deferoxamine, and dexrazoxane.

The agent-carrying component can also be a cyclodextrin. Cyclodextrin is a cyclic oligosaccharide with lipophilic inner cavity and hydrophilic outer surface capable of forming non-covalent inclusion complexes with a large variety of therapeutic agents with poor water solubility. Examples of the cyclodextrins include, but are not limited to, α-cyclodextrin, β-cyclodextrin, γ-cyclodextrin, hydroxyethyl-β-cyclodextrin, hydroxypropyl-β-cyclodextrin, methyl-β-cyclodextrin, dimethyl-β-cyclodextrin, randomly dimethylated-β-cyclodextrin, randomly methylated-β-cyclodextrin, carboxymethyl-β-cyclodextrin, carboxymethyl ethyl-β-cyclodextrin, diethyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, di-O-hexanoyl-β-cyclodextrin, glucosyl-β-cyclodextrin, and maltosyl-β-cyclodextrin.

### Therapeutic Agent

The therapeutic agent described in the present invention includes any therapeutic agents that can be used for intravitreal injection. Examples of therapeutic agent include, but are not limited to, a therapeutic antibody or its fragment (Fab), a peptide; an anti-angiogenic factor, a growth factor, a cytokine; nucleic acid-like component, such as therapeutic DNA, RNA, siRNA or antisense oligonucleotide; and a small molecule such as steroid or its derivatives. In one example of the invention, the therapeutic agent is Fab.

The term “effective amount” as used herein, means that amount of a therapeutic agent to be included in a sustained release composition, which provides a therapeutically effective amount of the therapeutic agent to a subject when the composition is administered to the subject.

As used herein, the term “therapeutically effective amount” refers to that amount of a therapeutic agent that elicits a biological or medicinal response in the eye of a subject that is being sought by a researcher, veterinarian, medical doctor or other clinician.

One skilled in the art will recognize that the “effective amount” of a therapeutic agent to be included in a sustained release composition can vary depending upon factors, such as the presence of other components in the composition, the dose range of the composition, the degree of severity of the disease to be treated, etc. Standard procedures can be performed to evaluate the effect of the administration of the therapeutic agent to a subject, thus allowing a skilled artisan to determine the effective amount of the therapeutic agent to be included in a sustained release composition.

According to the invention, less than 10% of the therapeutic agent may be separated or released from a composition according to an embodiment of the invention in the blood plasma after one hour of incubation at 37° C. Also, the composition according to an embodiment of the invention may stably intercalate water-insoluble therapeutic agent, so that less than 10% of the therapeutic agent may be separated or released from the nanosphere in the blood plasma after one hour of incubation at 37° C.

Furthermore, the present invention also provides a method of providing a sustained supply of a therapeutic agent to the eye of a subject in need thereof comprising:
providing a sustained release composition of the invention; and
intravitreally injecting the sustained release composition to a site of the eye of the subject.

The following examples illustrate the invention but are in no way intended to limit the scope of the present invention.

### EXAMPLES

### Example 1

Study of Radio-Labeled Antibodies, Antibody Fragments and Their Nanosphere Conjugates Locally Injected into Vitreous Humor in Rats

### Materials

Lipid raw materials including distearoylphosphatidylcholine (DSPC), cholesterol, and 1,2-distearoyl-sn-glycero-3-phosphoethanolamine-N-[maleimide(polyethylene glycol)2000] (DSPE-PEG2000-Maleimide) were obtained from NOF Corp. (Tokyo, Japan). N-(methoxy-(polyethylene glycol)-oxycarbonyl)-DSPE was purchased from Avanti Polar Lipids (Alabaster, Ala.).

### Preparation of Maleimide-PEG-DSPE Containing Nanosphere

A lipid mixture of 1,2-distearoyl-sn-glycerol-3-phosphocholine (DSPC), cholesterol, and 1,2-distearoyl-sn-glycero-3-phosphoethanolamine-N-[maleimide(polyethylene glycol)2000] (DSPE-PEG2000-Maleimide) (molar ratio 30:20:1.5) was dissolved in chloroform and then evaporated to dryness under vacuum by a rotary evaporator. The lipid film was re-suspended in MES buffer (100 mM MES, 260 mM NaCl and 2 mM EDTA, pH 5.5) at 62 to 65° C. to form a milky lipid suspension. The resulting lipid suspension was frozen and thawed 7 times, followed by repeated extrusion under argon 10 times through polycarbonate filters (Corning Nucleopore) of 200 nm pore size, 10 times through polycarbonate filters (Corning Nucleopore, WA, USA) of 200 nm pore size and 10 times through filters of 100 nm pore size using a pressure extruder (Lipex Biomembranes Inc., Vancouver, Canada) at 62° C. to 65° C. The final lipid concentration of the nanosphere was 101.2 μmol/mL and the mean particle diameter of the nanosphere is 104 (104±23) nm (determined by a dynamic laser particle sizer, N4+; Coulter Electronics, Hialeah, Fla., USA).

### Antibody Manipulation

Purified antibodies (12 mg per ml) were reduced by 2-mercaptoethylamine (MEA, final concentration is 0.05 M) for 90 min at 37° C. to produce thio-groups for the nanosphere conjugation (Yoshitake et al., Scand J Immunol. 1979; 10 (1):81-6). Antibody fragment were prepared by digesting whole antibody with pepsin (at a molar ratio of 4:1) and reduced by 2 mM of DTE for 30 min at 37° C. to produce thio-groups for the nanosphere conjugation. One hundred molecules of Fab were conjugated to each component of the nanosphere which contained Maleimide-PEG2000-DSPE on the surface. The efficiency of conjugation were evaluated by SDS-PAGE.

### Radiolabeling of ¹²⁵I

Protein was radiolabeled with ¹²⁵I using IODO-GEN® reagent (Pierce; IL, USA). Briefly, a protein sample was diluted with appropriate amount of phosphate buffer and carrier-free Na¹²⁵I was added to the reaction vessel. Typically, 500 μCi of Na¹²⁵I was mixed well with 25 μg protein and the reaction was allowed to proceed for 10 minutes at room temperature. At the end of the reaction time, reaction mixture was removed from the reaction vessel to terminate the iodination of the sample. The radiolabeling efficiency of all test materials is greater than 95 percent. The radiochemical purity was determined using thin layer chromatography (TLC), which was performed on a TLC aluminum sheet (Silica gel 60 F254; Merck, West Point, Pa.), with ethyl acetate/ethanol (85:15 [vol/vol]) serving as the mobile phase. The chromatograms were recorded using an imaging scanner (system 200; Bioscan).

### Animal Model

Male Brown Norway (BN) pigmented rats weighing of about 150 to 200 grams were used. The animals were handled in accordance with the Association for Research in Vision and Ophthalmology (ARVO) statement for the Use of Animals in Ophthalmic and Vision Research. They were anesthetized with intramuscular injections of 1.5 ml/kg of an equal volume mixture of 2-(2.6-xylidino)-5.6-dihydro-4H-1.3-thiazine-hydrochloride methylparaben (Rompun; Bayer AG, Leverkusen, Germany) and 50 mg/ml ketamin (Ketalar; Parke-Davis, Morris Plains, N.J., USA). After anesthesia, the pupils were dilated with 1% tropiamide (Mydriacyl; Alcon Laboratories, Hempstead, UK), and the eyes were gently protruded using a rubber sleeve. The eyes were then covered with a small piece of transparent sheet (3M, Minneapolis, Minn.) approximately 3 mm in diameter at the cornea by sodium hyaluronidase (Healon; Pharmacia and Upjohn) which served as a contact lens, allowing the fundus to be visible under a surgical microscope. A 90-degree periotomy was made in the temporal quadrant, and a sclerotomy was made 1 mm behind the limbus with the tip of a 27-gauge needle. A 33-gauge blunt-tip needle (Hamilton, Reno, Nev., USA) was inserted into the vitreous cavity, and 5 μl of the sample suspension containing 8 μg protein (equal to 150 μCi of isotope units) was injected. The needle was left in the vitreous cavity for 1 min after the injection to reduce the degree of reflux. The contra lateral eye was left untreated to provide the control for comparison.

### Single-Photon Emission Computed Tomography (SPECT) Imaging

SPECT for each rat was performed at 0.083 (2 hours), 1, 2, 7, and 10 days after 150 μCi of whole antibody, Fab, and Fab-nanosphere injection. At indicated time, the rat was anesthetized with intramuscular injection of 0.15 ml/kg of phenobarbital and SPECT imaging was carried out on an e.Cam Multiangle Cardiac (Siemens, Munich, Germany) equipped with a pinhole collimator. The center field of the view was 25.4 cm² and a single energy centered window was used at 159 keV, with a width of 20%. A series of scans (22 min/frame×6) were obtained over a period of 15 min. Images were reconstructed in a 128×128 format from data with 32 projections distributed over 180° around the rat and a 40 second scan for each projection. The projections of each experiment were processed by reconstruction using filtered back projection, with a low-pass Butterworth filter of order 22.4 and cutoff frequency of 0.43. Each transverse image was reconstructed in a 128×128 array with a pixel size of 1.9×1.9 mm and a zoom of 2.0.

### Retention Time Profile Study in Rabbit Vitreous Humor

The study was conducted according to the ARVO Statement for the use of Animals in Ophthalmic and Vision Research. New Zealand White rabbits received a single bilateral intravitreal injection dose of Fab or Fab-nanosphere. The eyes were collected and analyzed for the concentration of Fab and Fab-nanosphere in vitreous humor for up to 28 days after administration. Twenty-six New Zealand White rabbits, 0.9 to 1.6 kg on the day before drug administration, were assigned to two groups. Group 1 and 2 received a single bilateral Fab or Fab-nanosphere intravitreal injection dose of 75 μg per eye, through a 29-gauge needle. The rabbits were anesthetized by intramuscular injection of a mixture of 30 mg/kg ketamine and 10 mg/kg Xylazine. The Fab and Fab-nanosphere were then administered through the sclera and pars plans approximately 4 mm posterior to the limbus and the needle directed posterior to the lens into mid-vitreous. Rabbits per group were euthanized on days 1, 3, 7, 14, 21, and 28 and the eyes were enucleated. The vitreous humor were collected and stored at 4° C. Then, the samples were assayed for the concentration of Fab and Fab-nanosphere.

### Activity Measurement of Fab and Fab-Nanosphere by Competitive ELISA

The concentrations of Fab and Fab-nanosphere in vitreous humor were determined by competitive ELISA. In brief, this assay was performed by using anti-VEGF antibody to capture free His₆-VEGF and anti-penta-His conjugated HRP monoclonal antibody purchased from Quiagen for detection. The Vitreous humor was collected at various time points after the intravitreal injection. The concentrations of Fab and Fab-nanosphere in the vitreous humor were determined by competitive ELISA.

### Data Analysis:

The pharmacokinetics of Fab and Fab-nanosphere in vitreous humor were analyzed by a non-compartment method, using mean concentrations. All analyses were performed using SigmaPlot software.

The intravitreal injection is an efficient way to deliver therapeutic agents in eye diseases but it is an obstacle being an invasive administration for patients. To develop a sustained formulation for less frequent administration is beneficial to patients.

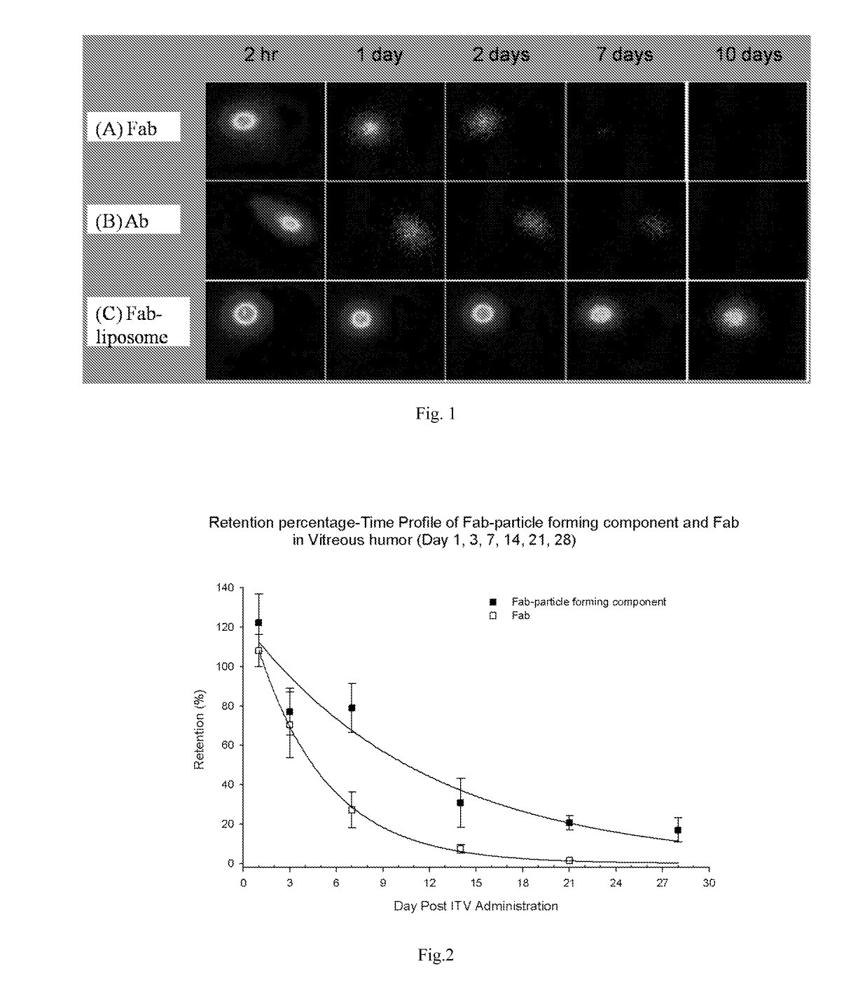

To compare the retention time of whole antibody, Fab, and Fab-nanosphere in the rat vitreous, each test sample of equal amount (8 μg protein/150 μCi) was applied through intravitreal injection to the eye. The SPECT images of the rats showed strong radiation activity in all test groups two hours after the injection. However, in the Fab and whole antibody groups, the radiation activity declined rapidly after one day and could not be detected after ten days as shown in FIG. 1. In contrast, Fab-nanosphere can retain in the vitreous humor much longer and a strong radioactivity was detected even at ten days post injection. The data indicate that Fab-nanosphere conjugated with the therapeutic agent is capable of providing a sustained release of the therapeutic agent more than 10 days at the disease sites.

To measure ocular pharmacokinetic of Fab and Fab-nanosphere activity in rabbit, the vitreous humor was collected and analyzed by competitive ELISA after 75 μg of Fab and Fab-nanosphere administration per eye as shown in FIG. 2. The peak vitreous concentration (Cₘₐₓ) of Fab and Fab-nanosphere group was 81.3 μg/ml versus 84.4 μg/ml, tₘₐₓ were both 1 d, t_{1/2} was 3.1 days versus 8.1 days, and the mean resident time (MRT) was 4.5 days versus 11.7 days. Noticeably, the biological activity in Fab-nanosphere group still retained 17% binding activity as 13 μg/ml of Fab after 28 days injection, but the activity in Fab group can not be measured in virtue of under the lower limit of detection. These results demonstrate that the Fab-nanosphere not only provides the sustained release property but is capable of maintenance of 17% biological activity until 28 days. It suggests that the therapeutic agent is able to accumulate at the disease site more than 28 days after the intravitreal injection.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method for treating an eye disease, comprising
intravitreally administering a composition comprising
a liposome derivatized with one or more hydrophilic polymers; and
a first therapeutic agent;
wherein the first therapeutic agent is covalently attached to the distal end of said hydrophilic polymer.

2. The method according to claim 1, wherein each hydrophilic polymer has a long chain neutral polymer attached to a lipid molecule.

3. The method according to claim 1, wherein the hydrophilic polymer is polyethylene glycol (PEG).

4. The method according to claim 2, wherein the hydrophilic polymer is 1,2-Distearoyl-sn-Glycero-3-Phosphoethanolamine-N-[Maleimide (Polyethylene Glycol)2000] (PEG-DSPE2000-maleimide).

5. The method according to claim 1, wherein after intravitreally administering the composition, the first therapeutic agent remains at the injection site for more than 7 days after the intravitreal injection.

6. The method according to claim 1, wherein after intravitreally administering the composition, the first therapeutic agent remains at the injection site for more than 28 days after the intravitreal injection.

7. The method according to claim 1, wherein the first therapeutic agent is selected from an antibody or its fragments (Fab), peptides, antiangiogenic factors, growth factors and cytokines, a nucleic acid-like component or a small molecule.

8. The method according to claim 7, wherein the small molecule is a steroid or its derivatives.

9. The method according to claim 7, wherein the nucleic acid-like component is selected from DNA, RNA, siRNA or antisense oligonucleotide.

10. The method according to claim 7, wherein the antibody is an anti-VEGF antibody.

11. The method according to claim 1, wherein the eye disease is confined to the posterior portion of the eye.

12. The method according to claim 1, wherein the eye disease is in close proximity to the retina.

13. The method according to claim 1, wherein the eye disease is age-related macular degeneration.

14. The method according to claim 1, wherein the eye disease is diabetic retinopathy.

15. The method according to claim 1, wherein the liposome comprises an agent-carrying component, wherein the agent-carrying component forms a complex with a second therapeutic agent.

16. The method according to claim 15, wherein the second therapeutic agent is selected from an antibody or its fragments (Fab), peptides, antiangiogenic factors, growth factors and cytokines, a nucleic acid-like component or a small molecule.

17. The method according to claim 16, wherein the small molecule is a steroid or its derivatives.

18. The method according to claim 16, wherein the nucleic acid-like component is selected from DNA, RNA, siRNA or antisense oligonucleotide.

19. The method according to claim 16, wherein the antibody is an anti-VEGF antibody.

20. The method according to claim 16, wherein the second therapeutic agent is selected from an antibody or its fragments (Fab), peptides, antiangiogenic factors, growth factors and cytokines, a nucleic acid-like component or a small molecule.
